(19) United States
(12) Patent Application Publication
(10) Pub. No.: US 2022/0409268 A1
(43) Pub. Date: Dec. 29, 2022

(54) MONOPOLAR PLASMA CURETTE ELECTROSURGICAL DEVICE

(71) Applicant: Medtronic Advanced Energy LLC, Minneapolis, MN (US)

(72) Inventors: Roger D. Greeley, Portsmouth, NH (US); Xiaoming Cheng, Keller, TX (US); Prakash Manley, Arvada, CO (US); Mohammad Miri, Longmont, CO (US); Bradley W. Jacobsen, Erie, CO (US)

(21) Appl. No.: 17/849,256

(22) Filed: Jun. 24, 2022

Related U.S. Application Data
(60) Provisional application No. 63/215,212, filed on Jun. 25, 2021.

(51) Int. Cl. A61B 18/14

(52) U.S. Cl. CPC A61B 18/1482; A61B 2018/00601

(57) ABSTRACT

An electrosurgical device including the disclosure describes an electrosurgical device including an elongated body having a tubular section extending from a proximal end to a distal end and defining an evacuation channel configured to evacuate tissue from the distal end to the proximal end, a curette at the distal end of the tubular section, wherein the curette defines a perimeter cutting edge that forms a distal opening to the evacuation channel, a plasma cutting electrode defined by the perimeter cutting edge of the curette, where the plasma cutting electrode is configured to operate in a monopolar configuration to deliver radio frequency (RF) plasma energy to adjacent tissue to cut a volume of the target tissue, and a dielectric coating on at least a portion of the curette, the dielectric coating electrically insulating the curette from target tissue and the volume of cut target tissue, wherein the dielectric coating comprises a ceramic material.

### CROSS-REFERENCE TO RELATED APPLICATIONS

This application claims the benefit of U.S. application Ser. No. 63/215,212 filed Jun. 25, 2021, entitled “MONOPOLAR PLASMA CURETTE ELECTROSURGICAL DEVICE”.

### FIELD

This invention relates generally to surgical methods and apparatuses and particularly to electrosurgical devices.

### BACKGROUND

Electrosurgical devices such as plasma-mediated thermo-electric cutting devices have been developed for use in cutting soft biological tissue in surgical settings. Such devices have found use in various surgical settings and procedures including, but not limited to, spine discectomy and fusion, and other surgical specialties such as general surgery, breast, thoracic, and the like. Typically, such electrosurgical devices are classified as being either monopolar or bipolar electrosurgical devices. A monopolar device generally includes a single electrode carried by the device and configured to communicate with a reference electrode, typically in the form of a return pad, attached to the exterior of a patient. Monopolar electrosurgical devices deliver highly concentrated electrical energy that enhances cutting edges to excise material and then transmits through the tissue of a patient. In contrast, a bipolar electrosurgical device includes a pair of electrodes carried by the device and positioned in close proximity to one another.

### SUMMARY

The techniques of this disclosure generally relate to electrosurgical cutting devices with a monopolar plasma electrode at the distal end of the device. More specifically, the disclosed devices include a curette style tip with the perimeter edge of the curette defining the monopolar plasma electrode. The device may further include an evacuation system that can be operated in conjunction with the electrosurgical plasma cutting process. The curette tip may be useful in certain electrosurgical procedures to scrape and remove excised tissue.

In an embodiment, the disclosure describes an electrosurgical device including an elongated body having a tubular section extending from a proximal end to a distal end and defining an evacuation channel configured to evacuate tissue from the distal end to the proximal end, a curette at the distal end of the tubular section, wherein the curette defines a perimeter cutting edge that forms a distal opening to the evacuation channel, a plasma cutting electrode defined by the perimeter cutting edge of the curette, where the plasma cutting electrode is configured to operate in a monopolar configuration to deliver radio frequency (RF) plasma energy to adjacent tissue to cut a volume of the target tissue, and a dielectric coating on at least a portion of the curette, the dielectric coating electrically insulating the curette from target tissue and the volume of cut target tissue, wherein the dielectric coating comprises a ceramic material.

In another embodiment, the disclosure describes an electrosurgical system including an electrosurgical device including an elongated body having a tubular section extending from a proximal end to a distal end and defining an evacuation channel configured to evacuate tissue from the distal end to the proximal end, a curette at the distal end of the tubular section, where the curette defines a perimeter cutting edge that forms a distal opening to the evacuation channel, a plasma cutting electrode defined by the perimeter cutting edge of the curette, where the plasma cutting electrode is configured to operate in a monopolar configuration, a dielectric coating on at least a portion of the curette, the dielectric coating electrically insulating the curette from target tissue and the volume of cut target tissue, where the dielectric coating comprises a ceramic material, a reference electrode, and a power supply coupled to the electrosurgical device and reference electrode, where the power supply is configured to deliver radio frequency (RF) plasma energy to the plasma cutting electrode to cut a volume of the target tissue.

In another embodiment, the disclosure describes a method of producing a coring electrode for an electrosurgical device, the method including providing an elongate body having a tubular body and a curette at a distal end of the tubular body, where the curette and tubular body comprise a metal substrate, where the elongated body comprises an inner surface and an outer surface, the inner surface defining an evaluation lumen that extends from the distal end to a perimeter cutting edge defined by the curette; applying a ceramic material on an inner and an outer surfaces of the curette to form a dielectric coating; wherein the metal substrate is sufficiently exposed at the perimeter cutting edge to define a plasma cutting electrode configured to deliver radio frequency (RF) plasma energy to adjacent tissue in a monopolar configuration.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The disclosure can be more completely understood in consideration of the following detailed description of various embodiments of the disclosure, in connection with the accompanying drawings, in which:

FIG. 1 is a schematic view of an example electrosurgical device that includes a curette with a plasma cutting electrode tip.

FIGS. 2A and 2B are schematic cross-sectional views of curette of FIG. 1 illustrating cutting features of a plasma cutting electrode.

FIGS. 3A and 3B are schematic views of another electrosurgical device that includes a curette plasma cutting electrode with an articulating tip.

FIG. 4 is a flow diagram of an example method of producing the disclosed curette plasma cutting electrodes.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

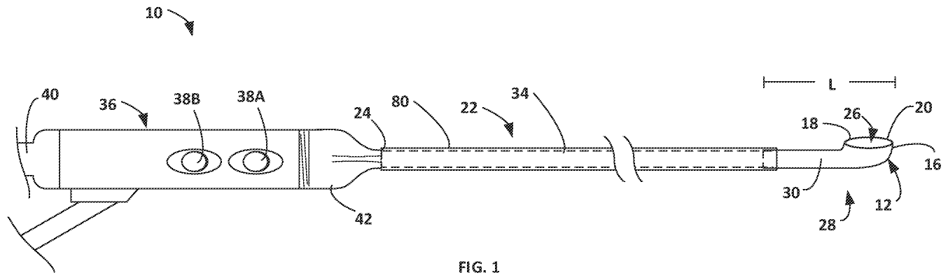

FIG. 1 is a schematic view of an example electrosurgical device 10 having a curette 12 at the distal end 16 of the device with a perimeter cutting edge 18 that defines plasma electrode 20. Device 10 may be used with electrosurgical procedures including, but not limited to, performing end plate preparation steps of a discectomy, an entire discectomy via various approaches, or microdiscectomies. Curette 12 defines a scoop-shape that allows for device 10 to easily cut and separate volumes of target tissue through simple rotation of the device and dislodge the cartilaginous endplate that protects the cortical bone surface of adjacent vertebral bodies. This may be particularly suitable for discectomy procedures, which involve the surgical removal of an intervertebral disc and fusion of adjacent vertebra. Intervertebral discs are flexible pads of fibro cartilaginous tissue tightly fixed between the vertebral bodies of the spine. The discs comprise a flat, circular capsule roughly 1 to 2 inches in diameter and about 0.25 to 0.5 inches thick and made of a tough, fibrous outer membrane called the annulus fibrosus, surrounding an elastic core called the nucleus pulposus. Under stress, it is possible for the annulus fibrosus to fail or the nucleus pulposus to swell and herniate, pushing through a weak spot in the annulus fibrosus membrane of the disc and into the spinal canal. Consequently, all or part of the annulus fibrosus and/or nucleus pulposus material may protrude through the weak spot, causing pressure against surrounding nerves which results in pain and immobility.

Where a damaged intervertebral disc must be removed from the patient as part of a discectomy and a subsequent fusion of vertebral bodies of the superior and inferior vertebrae, the surgeon may first retract soft tissue from the point of entry to the vertebrae to be fused. Around and attached to the vertebrae are, among other things, various muscles which act on the vertebrae to affect movement of the upper body. Once the retraction is complete, and the disc is exposed, the disc may be removed. The vertebrae may then be aligned to straighten the spinal column, and stabilized relative to one another by rods or other supports which are attached to the vertebrae by numerous fastening techniques. The surgeon may then place implants and bone grafts across the exposed surfaces of adjoining vertebrae and restore the location of the soft tissue to cover the bone grafts and vertebrae. The grafts regenerate, grow into bone and fuse the vertebrae together, with the implant and rod functioning as a temporary splint which stabilizes the spinal column while the bone fuses together over a period of months.

During the discectomy, the disclosed devices may be particularly useful to separate and remove volumes of intervertebral disc without significant damage to the surrounding tissue. The devices may generate monopolar radio frequency (RF) plasma. The plasma may cut and separate the target tissue. Additionally, the shape, and sharpness of perimeter edge 18 may further help cut and remove the target tissue. The general shape of curette 12 may also assist with removal of tissue in limited access locations where blades or other electrosurgical devices may have difficulty removing such tissue. In some examples, the devices may also be used to shrink and seal blood vessels of the vertebral venous or arterial systems against blood loss before or after the vessels are cut, rupture or are otherwise severed.

Electrosurgical device 10 includes an elongated body 22 extending from a proximal end 24 to a distal end 16. Elongated body 22 may define an inner lumen configured to evacuate and remove excised tissue from distal end 16 toward proximal end 24 (evacuation channel 26). Distal end 16 includes curette 12 having a scoop shape with perimeter cutting edge 18 that defines the entrance to evacuation channel 26 and forms plasma cutting electrode 20.

In some examples, to produce plasma cutting electrode 20, the distal portion 28 of elongated body 22 may be coated with a dielectric material (dielectric coating 30) configured to electrically insulate distal portion 28 of elongated body 22 from the surrounding patient tissue, the excised material being evacuate through evacuation channel 26, or both. As discussed further below, the dielectric coating 30 may be applied over the cutting tip that forms plasma cutting electrode 20 which either recedes during the curing process or becomes sufficiently thin that the electrode surface becomes defined by initial activation. In some examples, dielectric coating 30 may include a ceramic material applied to the inner and outer surfaces of curette 12 including a portion of elongated body 22. For example, elongated body 22 including curette 12 may be formed of an electrically conductive substrate 34 (e.g., stainless steel) such that the entire elongated body 22 and curette 12 act as the electrical conductors for plasma cutting electrode 20, with the actual plasma generating surface of the electrode being defined by the exposure of underlying electrically conductive substrate 34 along perimeter cutting edge 18. In such examples, dielectric coating 30 electrically insulates the portions of curette 12 and substrate 34 from adjacent patient tissue that might otherwise contact the inner or outer surfaces of elongated body 22 and curette 12 leading to an electrical short in electrosurgical device 10.

Electrosurgical device 10 may also include a handle assembly 36, which in turn is configured to couple to an electrosurgical power supply (not shown) that delivers the electric energy to plasma cutting electrode 20. The electrosurgical power supply may be configured to generate and provide radiofrequency (RF) monopolar energy based on the impedance parameters described herein. Handle assembly 36 may also include one or more switches or buttons 38A and 38B for activating plasma cutting electrode 20 to begin the desired electrosurgical energy to the surrounding tissue or to initiate suction for excavation of material through evacuation channel 26. Additionally, or alternatively, handle assembly 36 can include a stand or mount for stabilizing device 10 during an electrosurgical procedure. Handle assembly 36 may also include other switches or buttons for actuating other features of device 10, additional connectors for coupling device 10 to other components (e.g., coupler 40 for connecting to negative pressure pump and reservoir for excavation of material) during the procedure, and the like.

In some examples, elongated body 22 may be detachably coupled to handle assembly 36. For example, proximal end 24 of elongate body 22 may be attached to a connector 42 (e.g., screw or friction fit connector) that can be easily attached and detached from handle assembly 36 to allow for easy cleaning of elongated body 22 or interchange with other similarly configured elongated bodies having different geometries or configurations (e.g., larger or smaller diameter curette, different curette head angles, alternative perimeter cutting edge 18 shapes, or the like). Connector 42 may be sufficiently sized and textured for a clinician to grasp and detach elongated body 22 during the electrosurgical procedure. In such examples, connector 42 should be configured such that attachment to handle assembly 36 provides proper coupling between plasma cutting electrode 20 and the power source, to suction assemblies, sensor elements, and the like.

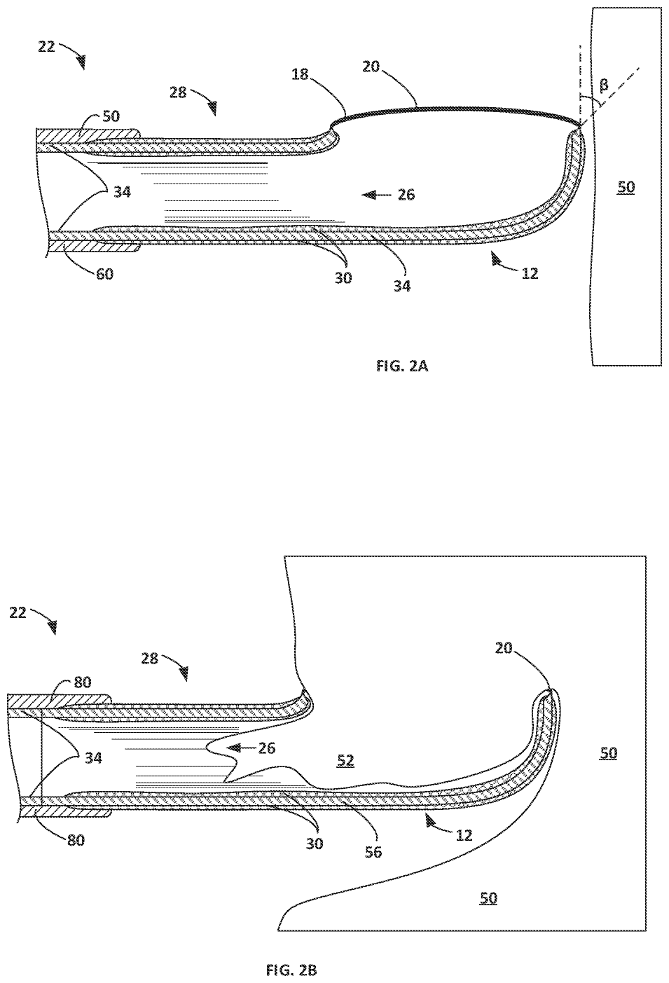

Plasma cutting electrode 20 is carried by distal portion 28 of elongated body 22 and configured to deliver electric energy (e.g., RF plasma, including a pulsed electron avalanche plasma, or ablation energy) to surrounding patient tissue (e.g., soft tissue or disc material) to cut a volume of the tissue at a target treatment site which is then conveyed into evacuation channel 26. FIGS. 2A and 2B are schematic cross-sectional views of distal portion 28 illustrating cutting features of plasma cutting electrode 20 during operation. The configuration of perimeter cutting edge 18 and plasma cutting electrode 20 define the entrance opening to evacuation channel 26. As electric energy is delivered to the surrounding soft tissue 50, plasma cutting electrode 20 cuts the surrounding tissue 50 to create a volume of tissue (e.g., excised tissue 52) that enters through the opening defined by perimeter cutting edge 18 and plasma cutting electrode 20 and is conveyed into evacuation channel 26.

The opening defined by perimeter edge 18 of curette 12 defines the entry to evacuation channel 26. As electric energy is delivered to the surrounding soft tissue via plasma cutting electrode 20, the perimeter edge 18 cuts the surrounding tissue 50 to create a volume of tissue (e.g., excised tissue 52) that enters through the opening and is conveyed into evacuation channel 26. To help facilitate conveyance of excised tissue 52 along evacuation channel 26, proximal end 24 is coupled to a negative pressure source (e.g., via handle assembly 36) to provide suction and collection of excised material 52.

In some examples, to help facilitate removal of excised tissue 52, the surface of evacuation channel 26 may be coated or treated to prevent tissue adherence. Such coatings may include lubricious silicone materials or repellant omniphobic materials. Such treatments may include hydrophobic surface patternings. Excised tissue 52 may be separated from tissue 50 by various means including briefly pausing cutting motion and allowing the energy to cut across the inner diameter of electrode 20 or slightly rotating curette 12 to cut the base of excised tissue 52. The surrounding tissue 50 is preserved and remains relatively protected from thermal electrical energy due to dielectric coating 30 as the tissue passes along the exterior of curette 12.

Plasma cutting electrode 20 may be configured as a monopolar electrode configured to provide RF plasma to facilitate cutting of tissue 50. For example, plasma cutting electrode 20 may communicate with a reference electrode (not shown) also referred to as a return pad such as a back plate, dispersive pad, or topical pad connected to electrosurgical power supply to provide a monopolar arrangement. As used herein, the term “reference electrode” is used to signify an electrode configured to communicate with plasma cutting electrode 20 in a monopolar arrangement and is itself not carried by elongated body 22. The electric energy may be delivered in the form of radio frequency or pulsed radio frequency energy delivery to cut through surrounding soft tissue 50.

In a monopolar electrosurgical configuration, the active electrode, such as plasma cutting electrode 20, is positioned at the target surgical site. The reference electrode may be placed somewhere on the patient's body. Electrical current passes through the patient as it completes the electrical circuit from the active electrode to the reference electrode. The reference electrode has a much larger conductive surface area compared to the active electrode to help safely dissipate the electrical energy and prevent localized heating. In contrast, the active electrode has a much smaller surface area allowing for significant plasma or current to be produced at the treatment site to produce cutting, ablation, or coagulation affects. The electric current may be concentrated in the area of contact of the active electrode offering versatility and function with a variety of electrosurgical waveforms to produce different tissue effects. Monopolar electrosurgical configuration may be particularly useful in dry field plasma cutting and may be extended to wet field plasma cutting via waveform modification.

In some embodiments, particularly during use with discectomy procedures that include delicate surrounding tissue, to help decrease heat accumulation and associated collateral tissue damage, low voltage, current, power and/or low duty cycle waveforms may be used. Low power waveforms typically refer to low voltage, continuous waveforms. Low duty-cycle here typically refers to the proportion of time that the energy is actually being applied and may include cycles of less than 10% which may be, for instance, 1% or less, or 0.1% or less. A pulsed low duty-cycle signal may include a plurality of pulse bursts that are separated by more than one millisecond (e.g., has a frequency of less than 1 kHz) where each burst is shorter than one millisecond which may assist in minimizing tissue charring or burning.

The electrical signals suitable to create a plasma cutting effect are well known in the field. For instance, in the example of an applied RF (radio frequency) signal, the signal may have a frequency in the range of 200 kHz to 3.3 MHz applied in continuous, burst, or pulsed waveforms. Each burst typically has a duration in the range of 10 microseconds to 1 millisecond with each burst having a duty duration of about 0.1 to 10 microseconds. The pulses may be bi-phasic square waves that alternate positive and negative amplitudes. The interval between pulses should be shorter than a lifetime of the plasma vapor cavity in order to maintain the cavity and the plasma regime during each pulse burst. The time between the pulse bursts is sufficient so that the duty-cycle is relatively low. This minimizes the undesirable heating effects. Additional details regarding bipolar versus monopolar electrode selections and cutting versus coagulation operational parameters are described in U.S. Pat. No. 8,177,783 B2 entitled “Electric plasma-mediated cutting and coagulation of tissue and surgical apparatus,” the entire contents of which is incorporated by reference.

During operation plasma cutting electrode 20 as defined by perimeter cutting edge 18 forms the cutting edge of device 10. In some examples, the cutting edge may be mechanically sharp and produced by incorporating a beveled edge along perimeter 18. The beveled edge may provide a sharp leading edge that is beneficial in the application of dielectric coating 30 as discussed further below by helping to prevent the deposit of ceramic material at perimeter edge 18, thereby defining the surface area and location for plasma cutting electrode 20. Perimeter edge 18 may include a chiseled edge based on a set bevel angle (FIG. 2A, β) or may be further sharpened as desired by to incorporating a compound bevel, convex or hollow edge, v-edge, or the like. The relative sharpness of the perimeter edge 18 may help excise tissue 52 during application of electric energy but may not be sufficient to mechanically cut the surrounding tissue absent such application. The bevel angle may be about 10° to about 45°.

The size and shape of perimeter edge 18 along with the inner diameter of evacuation channel 26 may be any suitable size and may be tailored for a specified procedure. For example, the perimeter edge 18 may have an oval shaped diameter having a long axis on the order of about 2 mm to about 8 mm and a short axis diameter of about 2 mm to about 5 mm Additionally, or alternatively, the relative size of curette 12 may be sufficiently small, particularly for discectomy procedures to provide sufficient access during posterior lumbar interbody fusion (PLIF) or transforaminal lumbar interbody fusion (TLIF) surgery. In some examples, the inner diameter elongated body 22 (e.g., diameter of evacuation channel 26) may be on the order of at least 3 mm to ensure proper excavation of excised material 52 without producing an obstruction.

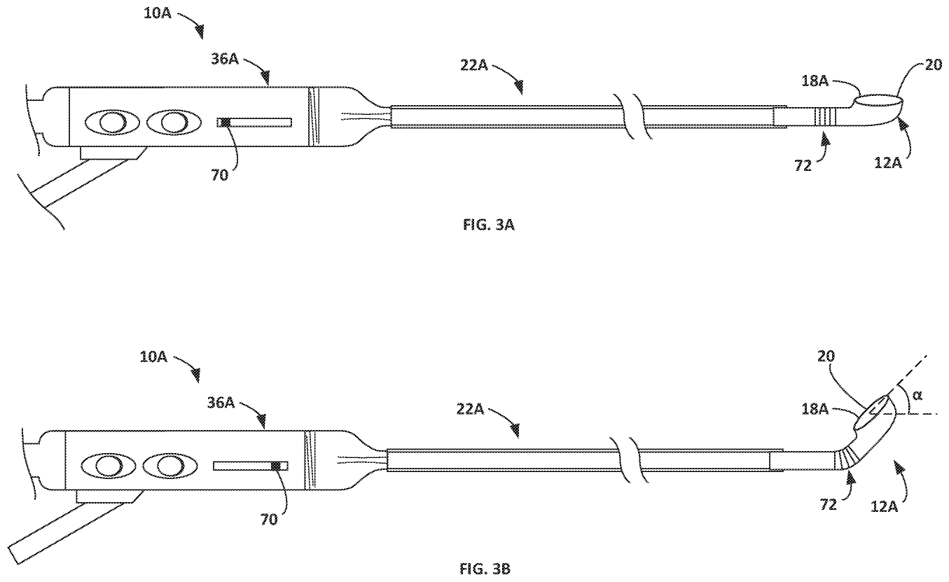

In some examples, perimeter edge 18 may be set substantially parallel to the longitudinal axis of elongated body 22 as shown in FIG. 1. Alternatively, perimeter edge 18 may be non-planar (e.g., curvilinear or undulating) or set at an offset head angle relative to elongated body 22. For example, FIGS. 3A and 3B are a schematic views of another electrosurgical device 10A having an elongated body 22A that includes a curette 12A having a perimeter edge 18A offset at a head angle (α) (FIG. 3B) relative to the longitudinal axis of elongated body 22A that may be used with the electrosurgical device 10. Having an offset head angle (α) can help with removing hard to reach tissue that may otherwise be inaccessible to reach using the 0° head angle configuration in FIGS. 1 and 3A. The head angle (α) may be fixed or variable.

In some examples, elongated body 22A may be configured with an articulating tip to produce head angle (α) for curette 12A. For example, handle assembly 36A of device 10A may include a lever 70 that allows the clinician to selectably adjust head angle (α) by rotation of curette 12A about articulation point 72. The articulation point 72 may be positioned proximal relative to perimeter edge 18A of curette 12 by a set distance tailored to a desired procedure (e.g., about 5 mm to about 20 mm from the proximal most edge of perimeter edge 18 for discectomy procedures). Articulation point 72 may be designed and implemented in a substantially similar manner to the articulation systems used with catheter assemblies, optical probes, and the like. Examples of articulation mechanisms for elongated bodies that may be incorporated into electrosurgical device 10A are described in U.S. Pat. No. 10,039,532 B2 entitled “Surgical Instrument with Articulation Assembly” by Srinivas et al.; U.S. Pat. No. 10,660623 B2 entitled “Centering Mechanism for Articulation Joint” by Nicholas; U.S. Pat. No. 10,561,419 B2 entitled “Powered End Effector Assembly with Pivotable Channel” by Beardsley; and U.S. Pat. No 8,403,946 B2 entitled “Articulating Clip Applier Cartridge” by Whitfield et al. all of which are incorporated by reference in their entirety.

Articulation point 72 may be actuated through handle assembly 36A through lever 70 which may include a slider mechanism. The articulating tip may allow the clinician to steer the distal end of elongated body 22A to allow plasma cutting electrode 20 to reach restrictive areas at the target treatment site without needing a direct line of sight access to the treatment location. Such articulation may be particularly useful for certain types of clinical procedures such as a discectomy. For example, in minimally invasive TLIF discectomies, access to the contralateral space of the disc may be constrained by tubular retractors or other similar access ports. With a straight electrosurgical device, full removal of nucleus material from the contralateral space may be impeded unless access is provided through both the left and right side of the spinal canal (like in a PLIF procedure). Even then, full removal of nucleus material located adjacent to the proximal wall of the spine between the two access points may be limited. Having an articulating tip may substantially improve removal of nucleus material and possibly reduce the number of access points needed to complete a particular procedure.

Alternatively, as opposed to inclusion of articulation point 72, elongated body 22A may include a fixed curve or bend. The fixed bend or curve may allow for convenient access to otherwise hard to reach treatment cites.

Plasma cutting electrode 20 may be composed of any suitable conductive material including, but not limited to, stainless steel, titanium, platinum, iridium, niobium or alloys thereof. In some examples, electrode 20 may be defined by an exposed surface of and underlying electrically conductive substrate material 56. The exposed surface may be formed by the application of dielectric coating 30 to the inner and outer surface of curette 12 such that perimeter cutting edge 18 of elongated body 22 is exposed to the surrounding tissue or includes a non-substantial amount of dielectric coating 30 thereby defining plasma cutting electrode 20.

The described surface exposure of electrode 20 may be the result of the application technique used for producing dielectric coating 30. For example, the dielectric coating 30 may be formed by applying a ceramic material and sintering (if needed) such material. In some examples, perimeter cutting edge 18, due to the edge's relative sharpness, may be partially exposed through dielectric coating 30 or include a minute layer of ceramic material such that the relative amount of coating created therefrom does not functionally impact the delivery of the RF plasma energy to the surrounding target tissue 50. The perimeter cutting edge 18 is sufficiently exposed to provide definition of plasma cutting electrode 20 while creating a sufficient dielectric coating 30 on the adjacent portions of substrate 56.

Dielectric coating 30 may include a ceramic material that produces an electrical barrier between underlying conductive substrate 56 of curette 12 and substrate 34 of elongated body 22 with respect to surrounding and excised tissue 50 and 52. Dielectric coating 30 may be applied to both the inner and outer surfaces of elongated body 22 and curette 12 using a suitable application technique to produce a non-porous dielectric coating. The inclusion of a ceramic (e.g., glass) dielectric coating ensures that the coating is capable of withstanding the high temperatures that can be produced by plasma cutting electrode 20 (e.g., possible temperatures in excess of 1000° C.) without melting, delaminating, or otherwise physically or electrically degrading during operation.

Any suitable ceramic material may be used to produce dielectric coating 30 provided the material can produce a film coating that has a dielectric strength of at least about 1000V, e.g., at least about 3000V. In preferred examples, the ceramic material should be selected to have a comparable coefficient of thermal expansion (CTE) with substrates 34 and 56 (e.g., stainless steel). For example, because the operation of plasma cutting electrode 20 can produce a large temperature gradient on the order of 1000° C. between distal end 16 and other portions of elongated body 22, large discrepancies between the CTE of substrates 34 and 56 and dielectric coating 30 can generate mechanical stress at the interface between substrate 34 and dielectric coating 30 which in turn can cause the coating to fail due to spallation, cracking, and the like. By selecting a dielectric coating 30 that possesses a CTE similar to that of substrate 34 and 56 (e.g., CTE values within ±10% of one another) ensures that mechanical stress along the interface between dielectric coating 30 and substrates 34 and 56 is sufficiently reduced to avoid one or more of the above-described complications. Suitable ceramic materials that may be used to produce dielectric coating 30 may include, but are not limited to, alumina, zirconia, alkaline borosilicate glass, alkaline earth borosilicate glass, silicate glass-ceramics and the like. In some examples where substrate 34 and 56 are stainless steel (CTE of approximately 11), dielectric coating 30 may include alkali and alkaline earth borosilicate glasses. Additionally, or alternatively, the ceramic material selected for dielectric coating 30 should be a medically safe or inert material.

Dielectric coating 30 may be applied over the entire length of curette 12 and elongated body 22 or only a set length (L) of distal portion 28. While in general, it may be preferable to electrically insulate the entire exterior and interior surface of curette 12 and elongated body 22, the extreme temperature fulgurations during operation may be localized to portions adjacent to perimeter edge 18. Thus, dielectric coating 30 comprising a ceramic material may extend over set length (L) of distal portion 28 along the inner and outer surfaces of substrate 34 and 56. The remaining exterior and interior surfaces of substrates 34 or 56 may be covered with a second dielectric material 80, such a shrink-wrap polymeric material, layer of parylene, or the like that is relatively inexpensive in terms of material and manufacturing costs while providing the desired dielectric characteristics. The second dielectric material 80 may have a relatively low melting or failure temperature that would otherwise make the material unsuitable if used within distal portion 28 near plasma cutting electrode 20. However, due to the separation distance (L) between the second dielectric material 80 and electrode 20, the localized temperature of the substrate material adjacent to perimeter cutting edge 18 may remain relatively low and within the operational parameters of second dielectric material 80. Additionally, or alternatively, second dielectric material 80 may be used to secure other components to elongated body 22 such as electrical conductors, actuating levers, and the like.

Substrates 34 and 56 may represent separate components that are subsequently coupled in a fixed or detachable arrangement during manufacturing of device 10. Providing the two substrates as separate components may be convenient for the ease in manufacturing of curette 12 and elongated body 22. For example, elongated body 22 may consist of a metal tube subsequently welded to curette 12. The two substrates 34 and 56 may be processed separately allowing for different coatings or treatments to be performed on each part. However, while substrates 34 and 56 are described as separate components that are subsequently coupled together, in some examples substrates 34 and 56 may be a single component fabricated as a single article.

Other components of device 10 can be fabricated from biologically acceptable materials suitable for medical applications, including but not limited to, electrically conductive metals, synthetic polymers, ceramics, and combinations thereof. Some such materials may include metals such as stainless steel alloys and titanium, thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, polyethylene terephthalate (PET), semi-rigid and rigid materials, elastomers, rubbers, thermoplastic elastomers, thermoset elastomers, elastomeric composites, rigid polymers including polyphenylene, polyamide, polyimide, polyetherimide, polyethylene, and combinations thereof.

In some examples, plasma cutting electrode 20, or alternatively another external electrode carried by elongated body 22 (not shown), may also be configured for use in a sensing capacity to measure or interrogate one or more properties of surrounding tissue 50. Such sensing may include measuring the impedance of the surrounding tissue 50 to determine if there is a significant change to the type of tissue (e.g., contact with bone or other tissue density than desired), the properties of the tissue (e.g., charring of the tissue), or complications with the system (e.g., a slow or loss of irrigation). The sensing capacity can enhance the safety capacity of device 10 by providing more accurate feedback of the surrounding tissue during use.

In some examples, electrosurgical device 10 may include or be configured to receive a camera to view and monitor progress of excised tissue 52 from the target treatment site. For example, handle assembly 36 and evacuation channel 26 may be configured to decouple from the negative pressure source and receive a borescope that is traversed through evacuation channel 26 toward distal end 16 to inspect the target treatment site. After inspection, the borescope can be removed and the negative pressure source reattached to continue the electrosurgical procedure.

The cross-section of elongated body 22 may take on any suitable shape and size as desired for particular applications. For example, elongated body 22 may possess a tubular body having a circular, semi-circular, oval, curvilinear, rectangular, trapezoidal, triangular, or some other multi-faceted cross-sectional shape. In some examples, it may be useful have a combination of curved and straight sides to provide the clinician with multiple edging options for excising tissue. Further the intersections between adjacent sides may themselves be curvilinear, abrupt resulting in distinct edge transitions, or a combination of curvilinear and abrupt transitions. The selection of cross-sectional shape of elongated body 22 may help improve tissue removal.

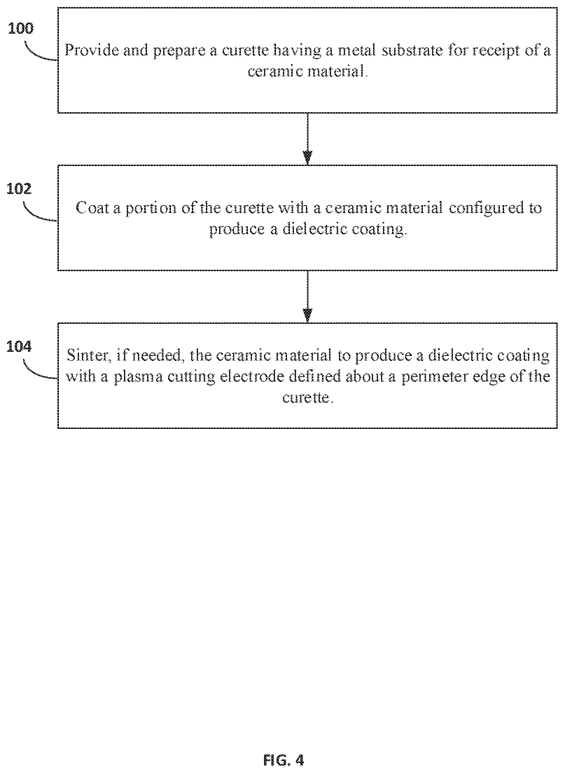

FIG. 4 is a block diagram of an example technique of producing plasma cutting electrode 20 on an electrosurgical device 10, described with respect to electrosurgical device 10 of FIG. 1. However the disclosed technique may be used to produce other electrosurgical devices or other techniques may be used to produce electrosurgical device 10.

The technique of FIG. 4 include providing and preparing a curette 12 having a metal substrate for receipt of a ceramic material (100), coating at least a portion of the curette 12 with a ceramic material configured to produce a dielectric coating (102), and sintering, if needed, the ceramic material to produce a dielectric coating 30 with a plasma cutting electrode 20 defined along a perimeter cutting edge 18 of curette 12 (104).

As discussed above, dielectric coating 30 may be applied to curette 12 and portions of elongated body 22 (e.g., inner and outer surfaces of the tubular body). Curette 12 may be initially coupled to elongated body 22 or may be coated separate of elongated body 22 and subsequently coupled thereto. To help assist with the application of the ceramic material, substrate 56 of curette 12, and optionally portions of substrate 34 of elongated body 22, may be initially optionally cleaned, chemically etched or treated, or the like. Such treatment may help ensure proper adherence of the resultant dielectric coating 30 thereby reducing the likelihood of delamination, cracking, spallation, or other defects between substrate 34 or 56 and dielectric coating 30.

Once prepped, curette 12 may be coated with a ceramic material configured to produce dielectric coating 30 (102). Any suitable technique of coating may be use to apply the ceramic material.

Suitable ceramic materials include non-toxic materials such as borosilicate glass having a relatively high or upon sintering provide a coating with a relatively high dielectric constant. The particle size of the ceramic material may be on the order of about 20 μm.

While the portions of substrate s 34 or 56 receiving the ceramic material and thereby dielectric coating 30 may be any suitable length including the entire length of elongated body 22, dielectric coating 30 should be applied to substrate 34 and 56 along the distal portion for a length of at least 5 mm as measured from the proximal most end of perimeter cutting edge 18 such that curette 12 is fully coated. Having the coating applied over at least such a length can help ensure proper electrical insulation between plasma cutting electrode 20 and surrounding tissue including excised tissue 52. While the outer surface of elongate body 22 may also receive a second dielectric coating 80, such a coating may not be configured to withstand the localized high temperatures near plasma cutting electrode 20, hence the minimal length of dielectric coating 30 can ensure the presence of a dielectric coating 30 near perimeter cutting edge 18 that can withstand the large temperature fluctuations.

The method of FIG. 4 also includes and sintering, if needed, the ceramic material to produce a dielectric coating 30 with a plasma cutting electrode 20 defined along perimeter cutting edge 18 of curette 12 (104). The sintering process may include heating substrate 34 under pressure to coalesce the ceramic particles to form a non-porous coating. The resulting dielectric coating 30 should have a dielectric strength of at least about 1000 V, e.g., at least 3000 V. Dielectric coating 30 may have a thickness of about 2-4 mils (e.g., about 0.05 mm to about 0.1 mm)

Various embodiments of systems, devices, and methods have been described herein. These embodiments are given only by way of example and are not intended to limit the scope of the claimed inventions. It should be appreciated, moreover, that the various features of the embodiments that have been described with respect to the different figures may be combined in various ways to produce numerous additional embodiments. For example, variations of the different electrodes may be combined with other internal electrodes, plasma cutting electrodes, external electrodes, and combinations thereof to produce an electrosurgical device tailored for a particular application or procedure. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. An electrosurgical device comprising:
an elongated body comprising:
a tubular section extending from a proximal end to a distal end and defining an evacuation channel configured to evacuate tissue from the distal end to the proximal end;
a curette at the distal end of the tubular section, wherein the curette defines a perimeter cutting edge that forms a distal opening to the evacuation channel;
a plasma cutting electrode defined by the perimeter cutting edge of the curette, wherein the plasma cutting electrode is configured to operate in a monopolar configuration to deliver radio frequency (RF) plasma energy to adjacent tissue to cut a volume of the target tissue; and
a dielectric coating on at least a portion of the curette, the dielectric coating electrically insulating the curette from target tissue and the volume of cut target tissue, wherein the dielectric coating comprises a ceramic material.

2. The electrosurgical device of claim 1, and wherein the plasma cutting electrode is configured to provide (RF) plasma energy in the range of about 10 W to about 250 W.

3. The electrosurgical device of claim 1, wherein the tubular section comprises an external surface and an interior surface defining the evacuation channel, and wherein the dielectric coating is applied to portions of both the external surface and the interior surface of the tubular body.

4. The electrosurgical device of claim 1, wherein the curette comprises a metal substrate configured to provide electrical conductivity to plasma cutting electrode.

5. The electrosurgical device of claim 1, wherein the tubular body and the curette comprise an electrically conductive metal, and wherein exposure of the conductive metal at the perimeter cutting edge defines the plasma cutting electrode.

6. The electrosurgical device of claim 1, wherein the dielectric coating has a coefficient of thermal expansion of about 8 ppm to about 15 ppm and a dielectric strength of at least about 1000V.

7. The electrosurgical device of claim 1, wherein the dielectric coating has a coefficient of thermal expansion that is within ±10% of a coefficient of thermal expansion of a metal substrate forming the curette.

8. The electrosurgical device of claim 1, wherein the dielectric coating comprises alkaline earth borosilicate glass.

9. The electrosurgical device of claim 1, wherein the dielectric coating comprises a non-porous film configured to withstand temperatures of at least about 800° C. without melting.

10. The electrosurgical device of claim 1, further comprising a second dielectric coating applied over an exterior surface of the elongated body, the second dialectic coating at least partially overlapping the dielectric coating on the curette or the tubular body.

11. The electrosurgical device of claim 1, where the perimeter cutting edge defines a bevel angle of about 10° to about 45°.

12. The electrosurgical device of claim 11, wherein the perimeter cutting edge comprises at least one of a chiseled bevel, a compound bevel, convex bevel, hollow bevel, or v-edge bevel.

13. The electrosurgical device of claim 1, wherein the elongated body is configured to receive a borescope through the evacuation channel for visualization of the target treatment site.

14. The electrosurgical device of claim 1, wherein the perimeter cutting edge has a head angle of about 0° to about 60° as measured relative to a longitudinal axis of the tubular body.

15. The electrosurgical device of claim 1, further comprising an articulating tip configured to alter a relative position of the curette relative to the tubular body.

16. The electrosurgical device of claim 1, wherein the perimeter cutting edge defines a non-planar or undulating closed loop.

17. An electrosurgical system comprising:
an electrosurgical device comprising:
an elongated body comprising:
a tubular section extending from a proximal end to a distal end and defining an evacuation channel configured to evacuate tissue from the distal end to the proximal end;
a curette at the distal end of the tubular section, wherein the curette defines a perimeter cutting edge that forms a distal opening to the evacuation channel;
a plasma cutting electrode defined by the perimeter cutting edge of the curette, wherein the plasma cutting electrode is configured to operate in a monopolar configuration; and
a dielectric coating on at least a portion of the curette, the dielectric coating electrically insulating the curette from target tissue and the volume of cut target tissue, wherein the dielectric coating comprises a ceramic material;
a reference electrode; and
a power supply coupled to the electrosurgical device and reference electrode, wherein the power supply is configured to deliver radio frequency (RF) plasma energy to the plasma cutting electrode to cut a volume of the target tissue.

18. The electrosurgical system of claim 17, further comprising a negative pressure source coupled to the electrosurgical device, the negative pressure source configured to draw and collect tissue from the proximal end to the distal end of the elongated body.

19. A method of producing a coring electrode for an electrosurgical device, the method comprising:
providing an elongate body comprising a tubular body and a curette at a distal end of the tubular body, wherein the curette and tubular body comprise a metal substrate, wherein the elongated body comprises an inner surface and an outer surface, the inner surface defining an evaluation lumen that extends from the distal end to a perimeter cutting edge defined by the curette; and
coating at least a portion of the curette with a ceramic material on an inner surface and an outer surface of the curette to form a dielectric coating; wherein the perimeter cutting edge of the curette is sufficiently exposed to define a plasma cutting electrode configured to deliver radio frequency (RF) plasma energy to adjacent tissue in a monopolar configuration.

20. The method of claim 19, wherein the dielectric coating has a coefficient of thermal expansion of about 8 ppm to about 15 ppm and a dielectric strength of at least about 1000V.

21. The method of claim 19, wherein the dielectric coating has a coefficient of thermal expansion that is within ±10% of a coefficient of thermal expansion of a metal substrate forming the curette.

22. The method of claim 19, wherein the perimeter cutting edge defines a bevel angle of about 10° to about 45°.

23. The method of claim 19, further comprising applying a second dielectric coating over the external surface of the elongated body, wherein the second dielectric coating overlaps with the dielectric coating and a distal end of the second dielectric coating is at least about 5 mm away from a proximal most portion of the perimeter cutting edge.
